(19) United States
(12) Patent Application Publication
(10) Pub. No.: US 2025/0009674 A1
(43) Pub. Date: Jan. 9, 2025

(54) GRANULES OBTAINABLE BY CONTINUOUS MELT GRANULATION

(71) Applicant: DSM IP ASSETS B.V., Heerlen (NL)

(72) Inventors: Bram BEKAERT, Kaiseraugst (CH); Christoph PORTIER, Kaiseraugst (CH); Lise VANDEVIVERE, Kaiseraugst (CH); Chris VERVAET, Kaiseraugst (CH)

(21) Appl. No.: 18/709,330

(22) PCT Filed: Nov. 15, 2022
(86) PCT No.: PCT/EP2022/081966

§ 371 Date: May 10, 2024

(30) Foreign Application Priority Data
Nov. 16, 2021 (EP) 21208530.2
Nov. 16, 2021 (EP) 21208535.1

(51) Int. Cl. A61K 9/50; A61K 31/07; A61K 31/122; A61K 31/355; A61K 31/375; A61K 31/4415; A61K 31/51; A61K 31/525; A61K 31/593; A61K 31/714; A61K 47/26

(52) U.S. Cl. CPC A61K 9/5089; A61K 9/5015; A61K 31/07; A61K 31/122; A61K 31/355; A61K 31/375; A61K 31/4415; A61K 31/51; A61K 31/525; A61K 31/593; A61K 31/714; A61K 47/26

(57) ABSTRACT

The present invention relates to water-soluble or water-dispersible granules that are obtainable by continuous melt granulation. The granules comprise at least one binder, at least one filler and at least one active ingredients. Torque overload may be avoided when using a combination of the herein disclosed filler (e.g. mannitol or an HIMO) and binder (e.g. sorbitol).

### TECHNICAL FIELD

The present invention relates to water-dispersible powders for human consumption.

### BACKGROUND OF THE INVENTION

Fat-soluble active ingredients as such cannot be dissolved in water. It is therefore common practice to encapsulate fat-soluble active ingredients. Water-dispersible microcapsules comprising fat-soluble micronutrients (e.g. fat-soluble vitamins) are commercially available at DSM® Nutritional Products (Switzerland).

Microcapsules are often poorly flowable, mostly due to their small particle size. To obtain a flowable powder, microcapsules are granulated.

Granulation is a size enlargement process that is often done via wet granulation using a solvent (water or organic solvent) to initiate binding between solid particles (e.g. microcapsules). A drawback of wet granulation is the need of getting rid of the solvent at the end of the granulation process. In case of using water as solvent, a significant amount of energy is needed to evaporate water. A further drawback of wet granulation is the risk of hydrolysis of the active ingredient. In case of organic solvents, potentially harmful residues and/or negative environmental impacts are of concern.

Dry granulation and melt granulation are known alternatives for wet granulation. Melt granulation operates via similar principles as wet granulation but uses a molten binder as granulation fluid to establish liquid bridges between the particles to be granulated. When cooling to room temperature, the binder solidifies and forms bridges between individual powder particles to yield a solid end product with a granular structure.

Most often, melt granulation is done in a heated powder bed. This is a batch process: processing of subsequent batches must wait until the current is finished. The drawbacks of batch processes can be overcome by using a continuous process.

Continuous granulation is often done with extruders. Extrusion granulation can be done with solvent (“continuous wet granulation”) or with heat (“continuous melt granulation”).

Continuous melt granulation can be done in a extruder. A potential problem during any extrusion process is torque overload. When torque overload occurs, the transmitted torque exceeds the set torque point of the extruder's torque limiter. When this occurs, the frictional force is no longer strong enough to transmit the torque from the driving shaft to the driven member, and the driven member slips between the friction disks. When the torque surpasses the limit, torque limiters operate as a clutch to quickly disconnect the drive from the driven system, removing much of the inertial energy from the drive train-typically in milliseconds or fractions of milliseconds. In an ideal (i.e. stable/optimized) process, torque limiters are just a backup: the drive is rarely (if at all) disconnected from the driven system because torque overload should not occur.

There is a need for a process for continuous melt granulation with a reduced risk of torque overload. The obtained granules should be flowable, storage stable and/or water-dispersible. The amount of fines (i.e. non-granulated residues) should be low.

### SUMMARY OF THE INVENTION

The problems underlying the present invention are solved by continuous melt granulation of a mixture that comprises at least one active ingredient. Preferred active ingredients are water-soluble and water-dispersible (pro-) vitamins.

The continuous melt granulation process is preferably done in an extruder. In a preferred embodiment of the invention, a co-rotating twin-screw extruder continuously churns out free flowing granules; no die is needed at the end of the extruder [cf. FIG. 1 of N. Kittikunakorn et al., “Twin-screw melt granulation: Current progress and challenges”, International Journal of Pharmaceutics, 588, (2020), 119670].

In addition to the at least one active ingredient, the mixture of the invention further comprises at least one edible binder. During continuous melt granulation, the binder of the mixture is molten or at least softened.

The mixture of invention further comprises at least one edible filler. The amount of filler exceeds the amount of binder. In contrast to hot-melt extrusion, there is no need to melt the filler during continuous melt granulation. The melting temperature of the at least one edible filler is higher than the melting temperature of the at least one binder.

The method of the invention is a method of manufacturing granules by continuous melt granulation, wherein the mixture according to the invention is fed into an extruder, preferably into a twin-screw extruder. Torque overload is very effectively avoided when using mannitol as filler and sorbitol as binder.

A preferred mixture comprises mannitol, sorbitol and at least one active ingredient,
wherein the mixture comprises from 5 weight-% to 15 weight-% sorbitol, based on the total weight of the mixture, and
wherein the weight ratio between mannitol and sorbitol is from 5:1 to 9:1 and is preferably from 8:1 to 9:1, and
   wherein the at least one active ingredient is preferably a vitamin or a pro-vitamin.

In an embodiment, a mixture comprises an HMO, sorbitol and at least one active ingredient,
wherein the mixture comprises from 5 weight-% to 15 weight-% sorbitol, based on the total weight of the mixture, and
wherein the weight ratio between the HMO and sorbitol is 4:1 to 10:1, 5:1 to 10:1, 5:1 to 9:1, 6:1 to 9:1, 6.5:1 to 9:1, or from 7:1 to 9:1.

The granules of the invention comprise or consist of the mixture of the invention.

### BRIEF DESCRIPTION OF THE FIGURES

FIG. 1 shows monitored torque (Nm) during an unstable continuous melt granulation process. A fraction of the data is shown, starting at time point 700 s and ending at time point 800 s. A single torque spike can halt the entire process (i.e. torque overload). Time dependency of torque is therefore an indication of process stability. If the composition of granules is poorly chosen, torque overload can sometimes hardly be prevented.

FIG. 2 shows monitored torque (Nm) during a stable continuous melt granulation process. A fraction of the data is shown, starting at time point 700 s and ending at time point 800 s. There are no significant torque spikes which is an indication of process stability. There is no risk of torque overload. This process stability often correlates with consistency of the granule quality attributes.

### DETAILED DESCRIPTION OF THE INVENTION

The granules of the invention are obtainable by continuous melt granulation of a dry, edible mixture that comprises primary particles and at least two edible excipients. During continuous melt granulation, primary particles are agglomerated. Thus, the granule of the invention is preferably a unit formed of numerous particles. Primary particles of a granule are smaller than the granule.

Both edible excipients are preferably water-soluble or water-dispersible. The melting temperature of a first edible excipient is low enough to be molten or at least be softened during continuous melt granulation. When molten or softened, the first edible excipient establishes bridges between the primary particles. Said bridges then solidify at room temperature. Therefore, the first edible excipient mostly acts as a binder. In an embodiment, the first edible excipient is a polyol. In an embodiment, the first edible excipient is sorbitol.

The melting temperature of a second edible excipient is relatively high. The second edible excipient mostly acts as a filler. In an embodiment, the second edible excipient is mannitol. In an embodiment, the second edible excipient is a human milk oligosaccharide (HMO), e.g. 2′-O-fucosyllactose, or a mixture of human milk oligosaccharides, e.g. a mixture comprising mixture comprising 2′-fucosyllactose and difucosyllactose.

The granule of the present invention may comprise one kind of primary particles only or preferably more than one kind of primary particles. The primary particles of the granule of the invention preferably comprise or consist of an active ingredient. Examples of primary particles are vitamin C crystals, thiamine mononitrate crystals, niacinamide crystals and pyridoxine hydrochloride crystals. In case of fat-soluble active ingredients, the primary particles are preferably water-soluble or water-dispersible microcapsules that encapsulate a fat-soluble active ingredient. Such microcapsules can be obtained by spray-drying of an emulsion comprising the lipophilic active ingredient and at least one emulsifier.

The granules of the present invention are preferably water-soluble or water-dispersible. Compositions comprising or consisting of such granules may be suitable for preparing a beverage.

### Filler of the Invention

Fillers are excipients used to increase the volume of the granule of the invention. Fillers can have further functions. Some fillers (e.g. dietary fibers or human milk oligosaccharides) also have health benefits.

The granule of the invention is meant for human consumption. Toxic fillers and non-edible fillers in general are therefore excluded.

The granule of the invention is preferably water-soluble or water-dispersible. Fillers having a solubility of less than 1 g per 100 ml water or less than 0.5 g per 100 ml water or less than 0.1 g per 100 mL water are therefore not preferred.

Typically, the melting temperature of the filler is higher than the melting temperature of the binder. However, this does not exclude the possibility that the filler is also (partially) melted or softened during continuous melt granulation. The melting temperature of the filler is preferably at least 150° C., more preferably at least 155° C. and most preferably at least 160° C. and is preferably from 151° C. to 240° C., is more preferably from 160° C. to 240° C. and is most preferably from 160° C. to 180° C.

In an embodiment, the filler is inulin, a human milk oligosaccharide (HMO), or mannitol. In an embodiment, the HMO comprises 2′-fucosyllactose (2′-FL). In an embodiment, the filler is a mixture comprising 2′-fucosyllactose and difucosyllactose (DFL). In an embodiment, the filler comprises granulated standard inulin. Standard inulin has a mean degree of polymerization (DP) from 10 to 20. Not preferred fillers are microcrystalline cellulose and pregelatinized starch.

In an embodiment, the filler is mannitol. When using mannitol in combination with sorbitol, the risk that torque overload occurs during continuous melt granulation in an extruder may be prevented or at least reduced.

### Binder of the Invention

Binders are excipients used to hold the ingredients of a formulation together. To do so, the binder is melted or softened during continuous melt granulation. Typically, the melting temperature of the binder is lower than the melting temperature of the filler and often also lower than the melting temperature of any added active ingredient. The melting temperature of the binder is preferably less than 140° C., more preferably less than 130° C., even more preferably less than 120° C. and most preferably less than 110° C. The melting temperature of the binder is preferably from 50° C. to 110° C., is more preferably from 60° C. to 100° C. and is most preferably from 70° C. to 100° C.

The granule of the invention is preferable water-soluble or water-dispersible. Binders having a solubility of less than 1 g per 100 ml water or less than 0.5 g per 100 ml water or less than 0.1 g per 100 ml water are therefore not preferred.

In an embodiment, the binder is a polyol, a pectin, inulin, cellulose-based hydrophilic pharmaceutically acceptable excipients, povidone, copovidone, polydextrose, gums and/or co-processed pharmaceutically acceptable excipients. In an embodiment, the biner is sorbitol, ribose (e.g. D-ribose), mannitol, xylitol, erythritol, maltitol or isomalt. In an embodiment, the binder is apple pectin or beet pectin. In an embodiment, the binder is hypromellose (HPMC) or hydroxypropyl cellulose (HPC). In an embodiment, the binder is gum Acacia, Xanthan gum or Guar gum. In an embodiment, the binder is polyvinyl caprolactam-polyvinyl, acetate-polyethylene glycol graft co-polymer; the latter is commercially available as Soluplus®. In an embodiment, the binder is sorbitol that has preferably a melting temperature of 98° C. or less. Such sorbitol is commercially available from Roquette®.

In an embodiment, the binder is ribose (such as D-ribose), polyethylene glycol, sorbitol or xylitol. In an embodiment, the binder is preferably a polyol, is more preferably a sugar alcohol, is even more preferably sorbitol or ribose (e.g. D-ribose), and is most preferably sorbitol having a melting temperature of 98° C. or less. Such sorbitol is commercially available from Roquette®. Sorbitol is a stereoisomer of mannitol.

### Active Ingredient of the Invention

In an embodiment, the mixture of the invention comprises at least one water-soluble or water-dispersible active ingredient. Preferred active ingredients are water-soluble and water-dispersible vitamins such as vitamin B1, vitamin B2, vitamin B3, vitamin B6 and/or vitamin B12. Crystalline and/or amorphous particles consisting of ascorbic acid, thiamine mononitrate, niacinamide crystals or pyridoxine hydrochloride crystals are commercially available at DSM® Nutritional Products, Switzerland. Particles comprising riboflavin, cyanocobalamin or biotin are also commercially available at DSM® Nutritional Products, Switzerland. In a preferred embodiment, the mixture of the invention comprises at least two, preferably at least three, more preferably at least four and most preferably at least five water-soluble or water-dispersible vitamins. In a more preferred embodiment, the mixture of the invention comprises at least two, preferably at least three, more preferably at least four and most preferably at least five water-soluble or water-dispersible vitamins, but essentially no vitamin C. In this context, “essentially no vitamin C” may mean less than 5 weight-%, preferably less than 3 weight-% and most preferably less than 1 weight-% vitamin C, based on the total weight of the mixture. In an embodiment, the vitamin C is ascorbic acid or an edible ester thereof.

In an alternative embodiment, the mixture of the invention comprises multiple microcapsules. Microencapsulation is a protective technology for encapsulating solid or liquid active ingredients into microparticles with a diameter of e.g. 1-900 μm. Thus, the size of the granule depends inter alia on the size of the microcapsules: relatively large microcapsules result in relatively large granules. One advantage of microencapsulation lies in that the solid or liquid active ingredient is completely coated and isolated from external environment. Microencapsulation may render fat-soluble active ingredients water-dispersible or water-soluble. Spray-drying is a suitable method for the microencapsulation of fat-soluble active ingredients. Prior to spray drying, the fat-soluble active ingredient is mixed with at least one encapsulating agent. Typically, an emulsion is thereby provided. Gum Arabic is a commonly used encapsulating agent. Spray-drying is one of the most widely used microencapsulation techniques, since it provides rapid evaporation of water and maintains the low temperature in the particles. Spray-dried microcapsules comprising fat-soluble vitamins are commercially available at DSM® Nutritional Products, Switzerland. Vitamins A, D, E, and K are exemplary fat-soluble vitamins. It is rather uncommon to encapsulate a mixture of fat-soluble vitamins. Usually, fat-soluble vitamins are separately encapsulated. When providing a mixture of multiple fat-soluble vitamins, various kinds of microcapsules are mixed.

### Mixture of the Invention

The mixture of the invention is suitable for continuous melt granulation (i.e. without solvent). It is a powderous mixture that can be fed into an apparatus that is suitable for continuous melt granulation (e.g. an extruder).

In contrast to wet granulation, no solvent is needed when doing continuous melt granulation. Thus, the mixture of the invention comprises less than 10 weight-%, preferably less than 8 weight-%, more preferably less than 5 weight-% and most preferably less than 3 weight-% solvent, based on the total weight of the mixture. This applies not only, but in particular when the solvent is water. Thus, the preferred mixture of the invention comprises less than 10 weight-%, preferably less than 8 weight-%, more preferably less than 5 weight-% and most preferably less than 3 weight-% water, based on the total weight of the mixture.

In one embodiment, the mixture of the invention comprises or consists of a filler, a binder and at least one active ingredient. In an embodiment, the active ingredient is a water-soluble or water-dispersible vitamin. In an alternative embodiment, the mixture of the invention comprises or consists of a filler, a binder and microcapsules.

In an embodiment, the mixture of the invention comprises or consists of a filler, a binder and at least one active ingredient, wherein the filler is a human milk oligosaccharide or a mixture of human milk oligosaccharides, and wherein said at least one active ingredient is a water-soluble or water-dispersible vitamin. In an embodiment, the mixture of the invention comprises or consists of a filler, a binder and microcapsules, wherein the filler is a human milk oligosaccharide or a mixture of human milk oligosaccharides.

Fillers are needed for size enlargement; they increase the volume of the granule of the invention. The mixture of the invention comprises preferably from 40 weight-% to 95 weight-%, from 50 weight-% to 95 weight-%, from 60 weight-% to 90 weight-%, or from 70 weight-% to 90 weight-% of at least one filler, based on the total weight of the mixture. The mixture of the invention may comprise more than one filler. Preferably, however, the invention may comprise one filler only. In an embodiment, the filler is a polyol, more preferably a sugar alcohol, even more preferably a stereoisomer of sorbitol and is most preferably mannitol. In the context of the present invention, stereoisomers of the same polyol are non-identical sugar alcohols. In an embodiment, the filler is a human milk oligosaccharide or a mixture of human milk oligosaccharides, is more preferably 2′-O-fucosyllactose (2′-FL), is even more preferably crystalline or amorphous 2′-O-fucosyllactose (2′-FL), and is most preferably crystalline 2′-O-fucosyllactose.

Typically, the mixture of the invention comprises less binder than filler. The weight ratio between the filler and the binder is preferably from 4:1 to 10:1, 5:1 to 10:1, 5:1 to 9:1, 6:1 to 9:1, 6.5:1 to 9:1, or from 7:1 to 9:1. The mixture of the invention comprises preferably from 5 weight-% to 15 weight-%, more preferably from 6 weight-% to 14 weight-% and most preferably from 8 weight-% to 13 weight-% of at least one binder, based on the total weight of the mixture.

The mixture of the invention may comprise more than one binder. Preferably, however, the mixture of the invention comprises one binder only. Thereby, the binder is preferably a polyol, is more preferably a sugar alcohol, is even more preferably sorbitol or ribose, and is most preferably sorbitol. The preferred ribose is D-ribose.

In one embodiment, the mixture of the invention comprises at least one water-soluble or water-dispersible vitamin. In case the mixture of the invention also comprises vitamin C, the mixture of the invention comprises in total preferably from 1 weight-% to 20 weight-%, more preferably from 1 weight-% to 15 weight-%, even more preferably from 1 weight-% to 10 weight-% and most preferably from 1 weight-% to 8 weight-% water-soluble and/or water-dispersible vitamins, based on the total weight of the mixture. In case the mixture of the invention comprises essentially no vitamin C, the mixture of the invention comprises in total preferably from 0.1 weight-% to 15 weight-%, more preferably from 0.5 weight-% to 10 weight-%, even more preferably from 1 weight-% to 8 weight-% and most preferably from 1 weight-% to 5 weight-% water-soluble and/or water-dispersible vitamins, based on the total weight of the mixture. Exemplary water-soluble and/or water-dispersible vitamins are ascorbic acid and edible esters thereof (vitamin C), thiamin, riboflavin, niacin, vitamin B6 (pyridoxine, pyridoxal, and pyridoxamine), folacin, vitamin B12, biotin, and pantothenic acid.

In one embodiment, the mixture of the invention comprises microcapsules. When granulating the mixture of invention, there is a certain risk that some of the microcapsules might be damaged. This risk can be reduced by creating a low-shear environment in the extruder (e.g. by choosing suitable kneading elements). However, this risk can also be reduced by decreasing the concentration of microcapsules in the mixture of the invention. The mixture of the invention comprises from 0.1 weight-% to 55 weight-%, from 1 weight-% to 40 weight-%, from 1 weight-% to 30 weight-%, from 1 weight-% to 20 weight-%, from 5 weight-% to 20 weight-%, or from 2 weight-% to 10 weight-% microcapsules, based on the total weight of the mixture. Typically, the herein described microcapsules encapsulate at least one fat-soluble active ingredient. Preferred fat-soluble active ingredients are fat-soluble micronutrients such as fat soluble vitamins and pro-vitamins. Beta-carotene is an example of a fat-soluble pro-vitamin. In one embodiment, the mixture of the invention comprises one kind of microcapsules only. In a preferred embodiment, however, the mixture of the invention comprises various kinds of microcapsules. In case of various kinds of microcapsules, the mixture of the invention comprises preferably at least two, more preferably at least three, even more preferably at least four and most preferably at least five fat-soluble active ingredients. In one embodiment, the mixture of the invention comprises microcapsules comprising vitamin A, microcapsules comprising vitamin E, microcapsules comprising beta-carotene, microcapsules comprising vitamin D and/or microcapsules comprising vitamin K. In a preferred embodiment, the mixture of the invention comprises microcapsules comprising an ester of vitamin A (such as vitamin A acetate), microcapsules comprising an ester of vitamin E (such as vitamin E acetate), microcapsules comprising beta-carotene, microcapsules comprising vitamin D3 and/or microcapsules comprising vitamin K1.

### Granules of the Invention

Preferred granules are obtainable by continuous melt granulation (i.e. without solvent) of the mixture of the invention, preferably using a twin-screw extruder. Thus, the granule of the invention comprises or consists of the mixture of the invention.

The mixture of the invention comprises primary particles. Upon continuous melt granulation, bridges are formed between the mixture's primary particles. Thus, the granule of the invention is larger than the size of its primary particles. Preferred granules of the present invention have a mass median particles size D50 (volume based) from 0.5 mm to 6 mm, preferably from 1 mm to 5 mm, more preferably from 1.5 mm to 4.5 mm and most preferably from 2 mm to 4 mm, measured using dynamic image analysis. In case of crystals consisting of an active ingredient, granules of the present invention may comprise more than 100, more than 1000, more than 5000 or even more than 10000 crystals. In case of microcapsules, granules of the present invention may comprise more than 10, more than 100, more than 500 or even more than 1000 microcapsules.

Each of the granules may comprise one kind of active ingredient only. In a preferred embodiment, however, the granule of the invention comprises various kinds of active ingredients. In case of various kinds of active ingredients, the granule of the invention comprises preferably at least two, more preferably at least three, even more preferably at least four and most preferably at least five active ingredients. In one embodiment, the granule of the invention comprises vitamin B1, vitamin B2, vitamin B3, vitamin B6 and/or vitamin B12, but preferably no fat-soluble vitamins. In an alternative embodiment, the granule of the invention comprises microcapsules comprising vitamin A, microcapsules comprising vitamin E, microcapsules comprising beta-carotene, microcapsules comprising vitamin D, and/or microcapsules comprising vitamin K. In a preferred embodiment, the granule of the invention comprises microcapsules comprising an ester of vitamin A (such as vitamin A acetate), microcapsules comprising an ester of vitamin E (such as vitamin E acetate), microcapsules comprising beta-carotene, microcapsules comprising vitamin D3 and/or microcapsules comprising vitamin K1.

In one embodiment, the granule of the invention comprises a filler, a binder and at least one active ingredient,
wherein the mixture comprises from 5 weight-% to 15 weight-% binder, based on the total weight of the mixture, and
wherein the weight ratio between the filler and the binder is from 4:1 to 10:1, and
wherein the melting temperature of the binder is lower than the melting temperature of the filler, and
wherein the melting temperature of the filler is from 151° C. to 240° C.

The granule of the present invention is preferably water-soluble or water-dispersible. This can be achieved by selecting a binder that is water-soluble or water-dispersible, by selecting a filler that is water-soluble or water-dispersible, and by selecting water-soluble and/or water-dispersible active ingredients. In case of fat-soluble active ingredients, water-soluble and/or water-dispersible microcapsules are preferably selected.

### Method of the Invention

The method of the invention is continuous melt granulation and is preferably continuous twin-screw melt granulation. Differences between batch melt granulation and continuous twin-screw melt granulation are listed in Table 1 of N. Kittikunakorn et al., “Twin-screw melt granulation: Current progress and challenges”, International Journal of Pharmaceutics, 588, (2020), 119670. In a preferred embodiment of the invention, the herein disclosed dry, powderous mixture is fed into an extruder that is suitable for continuous melt granulation. Volumetric powder feeders are thereby not preferred. In a preferred method of the invention, the mixture of the invention is fed into the herein described extruder using a gravimetric powder feeder. Gravimetric powder feeders result in a controlled and consistent feeding process, keeping changes in powder properties and process deviations over time into consideration.

In the method of the invention, a twin-screw extruder is preferably being used. Particularly preferred are twin-screw extruders with corotating screws. The corotating screws of the preferred extruder are modular and can hence be configured in a variety of setups, resulting in various zones. The purpose of the first zone near the inlet of the extruder is transport. Transport zones are often referred to as conveying zones. One or more kneading zones can be present. The kneading zone is typically located between two conveying zones with preferably a shaping zone at the extruder outlet.

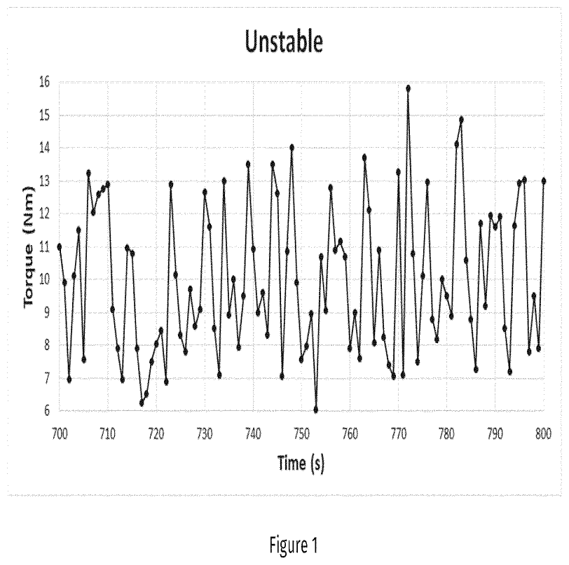

Most often, each zone has different screw elements. The conveying zone has conveying elements that transport material towards the granulator outlet [cf. section 2.1 of N. Kittikunakorn et al., “Twin-screw melt granulation: Current progress and challenges”, International Journal of Pharmaceutics, 588, (2020), 119670]. The kneading zone has kneading elements, e.g. narrower or wider kneading disks. A typical shaping zone has at least one size control element that minimizes the amount of oversized granules. An exemplary size control element is shown in FIG. 1(f) of J. Vercruysse et al. “Impact of screw configuration on the particle size distribution of granules produced by twin screw granulation”, International Journal of Pharmaceutics 479 (2015) 171-180. These size controlling elements are not knives as used for cutting extruded strands. Indeed, no spaghetti-like strands are extruded when doing continuous melt granulation. Extruders that are suitable for continuous melt granulation do not have a die at the outlet. Size control elements are screw elements within the extruder.

Hot-melt extrusion is different from the herein described continuous melt granulation. When doing hot-melt extrusion, strands with e.g. a cylindric diameter are extruded through a die. The length of the strand is not limited (i.e. could be endless). To obtain separated pellets, strands obtained by hot-melt extrusion need to be cut into pieces. The obtained pellets are not granules formed of distinguishable primary particles. When doing hot-melt extrusion, the cutting step can be done at any time after extrusion, including directly at the die of the extruder. Dies with an integrated knife are commercially available.

The above does not apply to the method of the present invention. When doing continuous melt granulation, no strand is extruded. Instead, granules are continuously churned out at the end of the extruder. Because no strand is produced, there is no need for a knife/cutting step, which significantly simplifies the process. When doing continuous melt granulation, a die at the end of the extruder is not needed. In a preferred embodiment of the invention, the mixture of the invention is fed into a twin-screw extruder that has no die and no knife cutting device.

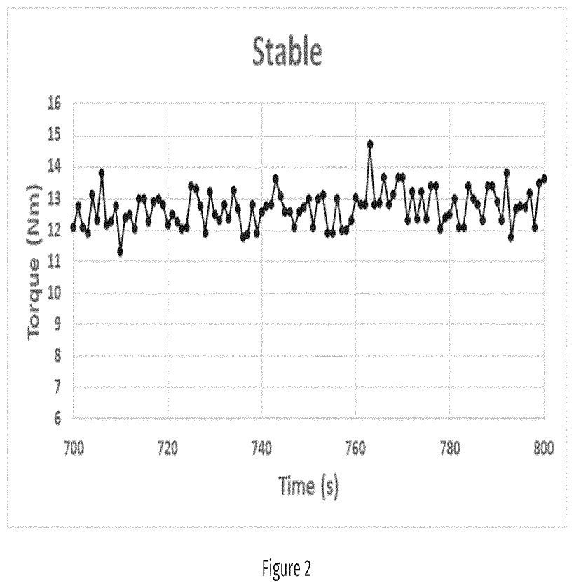

In the method of the invention, the screw configuration of a twin-screw extruder is typically selected such that the extruder has at least one kneading zone. Thereby, the at least one kneading zone is preferably closer to the powder inlet of the extruder than to the end of the extruder. Kneading zones have kneading elements. Said kneading elements are preferably kneading disks as disclosed in US 2005/0041521. Kneading disks may be congruent or non-congruent and are preferably positioned at a stagger angle from 30° to 90°. A stagger angle of approx. 30° is thereby preferred, as this is limiting the stress exerted on the powder mixture. In the context of the invention, “stagger angle” refers to the angle of crest misalignment that make two directly successive kneading disks, as explained in paragraph of US 2005/0041521. By way of example, the expression “kneading disks are positioned at a stagger angle 30°” means that there are successive kneading disks that make an angle of crest misalignment of 30°. There may be more than two successive kneading disks in a kneading zone of an extruder. FIG. 2 of US 2005/0041521 is a lateral view of a kneading zone with five successive kneading disks, wherein the kneading disks are positioned at a predetermined stagger angle.

Temperature control is relevant when doing continuous melt granulation. In a preferred method of the invention, the extruder has several zones which can be heated up or cooled down individually. When continuously melt granulating the herein disclosed mixture, temperature zones closer to the powder inlet of the extruder are typically heated. When choosing a suitable temperature, it must be taken into consideration that the material in the extruder may be moving rather quickly such that the contact of the material with the heating element is rather short. In some cases, it may therefore be advisable to set the temperature of some zones of the extruder to a temperature above the melting temperature of the mixture's binder.

In a preferred embodiment of the invention, the conveying zone and/or the kneading zone of the extruder are heated, such as to a temperature of from 90 to 210° C., from 90 to 200° C., from 100° C. to 190° C., from 100° C. to 200° C., from 150° C. to 210° C., from 160° C. to 200° C., from 120° C. to 190° C., or from 165° C. to 190° C.

In an embodiment, a gradual temperature decrease after having reached the hottest zone is applied, such as: conveying zone 1 (25° C.)-conveying zone 2 (170° C.)-kneading zone 3 (170° C.)-conveying zone 4 (55° C.)-conveying zone 5 (30° C.)-conveying zone 6 (25° C.)-shaping zone 7 (no temperature control).

Churning out hot granules is not preferred. Hot granules may still be relatively soft and sticky. As a consequence, hot granules may form a lump. This is to be avoided. It is therefore preferred to cool the material in the extruder before it is churned out by the extruder. In a preferred embodiment of the invention, at least one of the zones after the kneading zone is cooled down to a temperature of less than 60° C., preferably less than 40° C. and most preferably less than 26° C.

### EXAMPLES

### Example 1a (Premix—Fat-Soluble Vitamins)

In example 1a, a powderous premix comprising a mixture of microcapsules was provided. Each kind of microcapsule comprised a fat-soluble vitamin or a precursor of a fat-soluble vitamin. All microcapsules were obtained from DSM® Nutritional Products (Switzerland).

The premix provided in example 1a comprised vitamin A acetate, beta-carotene, vitamin D3, vitamin E acetate and vitamin K.

### Example 1b (Premix—Water-Soluble Vitamins)

In example 1b, a powderous premix comprising a mixture of water-soluble vitamins was provided. All vitamins were obtained from DSM® Nutritional Products (Switzerland).

The premix provided in example 1b comprised thiamin, riboflavin, niacin, vitamin B6, pantothenic acid, vitamin B12, biotin and optionally vitamin C.

### Example 2 (Ribose as Binder)

In example 2, a dry mixture comprising 47.13 weight-% inulin Orafti®GR (filler), weight-% D-ribose (binder) and 42.87 weight-% premix of example 1a, based on the total weight of the dry mixture, was provided. Thus, the weight ratio between filler and binder was 4.7:1.

Pre-experiments showed that the lowest possible processing temperature of D-ribose, still generating granules with desirable quality attributes, is approx. 80° C.

Orafti®GR is granulated inulin powder (average degree of polymerization≥10), available at Beneo, Mannheim, Germany). Its melting point has been determined in the range 190-195° C.

The dry mixture of example 2 was then fed into a ThermoFisher® Eurolab® extruder, using a gravimetric loss-in-weight feeder at the powder inlet. The extruder had a length-to-diameter (L/D) ratio of 25/1 and a screw diameter of 16 mm. The corotating screws of the extruder were fully modular and could hence be configured in a variety of setups. The extruder was segmented in several zones which can be heated up or cooled down individually.

In example 2, five extrusion experiments were run using various temperature schemes. Temperature zones closer to the powder inlet (zones 2, 3 and 4) were heated to temperatures from 104° C. to 130° C. The temperature of zones closer to the end of the extruder (zones 5 and 6) was kept at 25° C. Cooling the end of the extruder already enables material solidification and prevents stickiness of the granules that are being churned out of the extruder.

In example 2, the extruder had one kneading zone with three wide kneading disks that were positioned at a stagger angle of 60° (i.e. the angle of crest misalignment between any two directly successive kneading disks made 60° C.).

Reasonable granules were obtained in all five extrusion experiments. However, regardless of the applied temperature scheme, oil was oozing out of the obtained granules, indicating that some microcapsules were damaged during the extrusion process.

### Example 3 (Sorbitol as Binder)

In example 3, the process of example 2 was repeated. However, instead of D-ribose, sorbitol was used as binder. Pre-experiments showed that the lowest possible processing temperature of sorbitol, still generating granules with desirable quality attributes, is approx. 85° C. (i.e. slightly higher than D-ribose).

In example 3, five extrusion experiments were run. Temperature schemes and screw configurations similar to example 2 were applied.

Reasonable granules were obtained in all five experiments. Less oil was oozing out of the granules, indicating that sorbitol is performing better than ribose.

Different grades of sorbitol were then investigated in order to further reduce the energy required to generate good, water-soluble granules. Samples were analyzed via differential scanning calorimetry (DSC) in order to determine the sorbitol grade with the lowest melt energy. When using sorbitol grade Xtab300S (available at Roquette®) as a binder, the energy required to generate good water-soluble granules is lower than the other grades (cf. Table below).

| Sorbitol | Melt temperature | Melt Energy |
|---|---|---|
| Grade | (° C.) | (J/g) |
| Xtab300S | 97.84 | 126.5 |
| Xtab200S | 97.81 | 161.7 |
| P550SD | 97.86 | 165.1 |
| P650C | 99.73 | 174.9 |
| P300C | 99.64 | 180.3 |
| P100C | 99.57 | 181.9 |

### Example 4 (Effect of Binder on Properties of Granule)

In example 4, the effect of the binder on sphericity of the obtained granules was investigated. The following compositions were tested:

| | Temperature required |
|---|---|
| Binder | for forming granules |
| D-ribose | 80° C. |
| PEG 4000 | 85° C. |
| Sorbitol | 85° C. |
| Xylitol | 90° C. |
| Calcium lactate pentahydrate | 140° C. |

The granules having below compositions were then produced by continuous melt granulation:

| | Amount (wt.-%, | |
|---|---|---|
| | based on total | |
| Ingredient | weight of granule) | |
| Water-soluble vitamins | 8.1 | wt.-% |
| Binder | 10 | wt.-% |
| Inulin GR | 81.9 | wt.-% |
| Total | 100 | wt.-% |

In each case, granules were formed. However, differences in their sphericity were observed. The length-to-thickness (L/T) ratio, a sensitive measure of the sphericity of the granules, was measured on a Partan 3D (Microtrac Inc, Montgomeryville, PA, USA) particle size and shape analyzer using the method described in the operating manual (Version June 2017, Rev. I) and the results are outlined in below Table.

| Binder | L/T Ratio |
|---|---|
| D-ribose | 2.44 |
| PEG 4000 | 3.60 |
| Sorbitol | 2.11 |
| Xylitol | 2.81 |

PEG 4000 resulted in granules with the lowest sphericity with an L/T of 3.60. This demonstrates that PEG is not a good binder for forming spherical granules. Spherical granules are required for dosing in a micro-dosing packaging line. Sorbitol gave granules with the lowest L/T ratio (2.11). This indicates that sorbitol gave good spherical particles which could be dosed in a micro-dosing packaging line.

### Example 5 (Inulin-Torque Overload)

In example 5, the process of example 3 was repeated. However, instead of the premix of example 1a (fat-soluble vitamins), the premix of example 1b (water-soluble vitamins) was used. Good quality granules were obtained. However, only for a short period of time. Pretty soon after having started continuous melt granulation, torque overload occurred. It was not possible to find a stable process that could be run over a very long period of time (e.g. over a period of more than one hour). This jeopardizes the benefits of a process that is meant to be continuous.

### Example 6 (Mannitol as Filler)

In example 6, the process of example 5 was repeated. However, instead of inulin, mannitol (Pearlitol® 160 C; melting point approx. 165° C.) was used as filler.

First, a dry mixture comprising 83.65 weight-% mannitol (filler), 10 weight-% sorbitol (binder) and 6.35 weight-% premix of example 1b (including vitamin C), based on the total weight of the dry mixture, was continuously melt granulated in an extruder. In this first attempt, the weight ratio between filler and binder was approx. 8.4:1.

Secondly, a dry mixture comprising 87.91 weight-% mannitol (filler), 10 weight-% sorbitol (binder) and 2.09 weight-% premix of example 1b (no vitamin C), based on the total weight of the dry mixture, was continuously melt granulated in an extruder. In this second attempt, the weight ratio between filler and binder was approx. 8.8:1.

A screw configuration consisting of a single kneading zone with 3 kneading elements positioned at a stagger angle of 30° was used, similar to example 4. Temperature zones closer to the powder inlet (zones 2 and 3) were heated to temperatures from 170° C. to 180° C. The temperature of the zones closer to the end of the extruder (zones 4, 5 and 6) was gradually decreased: zone 4 (55° C.)-zone 5 (35° C.)-zone 6 (25° C.).

Both attempts were successful. Torque overload as observed in example 5 could be prevented by switching the filler (inulin->mannitol), which is beneficial to obtain a continuous process.

### Example 7 (Benefits of Mannitol/Sorbitol Combination)

In example 7, the benefits of using mannitol as a filler and sorbitol as a binder were confirmed, using the screw configuration and temperature zones of example 6.

In a first experiment of example 7, a placebo mixture consisting of mannitol (filler) and sorbitol (binder) only were continuously melt granulated in an extruder. In this placebo mixture, the weight ratio between filler and binder was 9:1. The first experiment was successful. No torque overload occurred.

In a second experiment of example 7, microcapsules comprising fat-soluble vitamins is continuously melt granulated using mannitol as a filler and sorbitol as a binder. Depending on the chosen fat-soluble vitamin, the weight ratio between filler and binder is from 7:1 to 8.9:1. No torque overload occurs, regardless of the chosen fat-soluble vitamin.

### Example 8 (HMO as Filler)

A dry mixture comprising 90 weight-% 2′-O-fucosyllactose (2′-FL) and 10 weight-% sorbitol, based on the total weight of the dry mixture, was provided. 2′-FL as obtained from DSM® Nutritional Products (Switzerland) comprises a minor amount of difucosyllactose (DFL).

The powderous mixture was then fed into a ThermoFisher® Eurolab® extruder, using a gravimetric loss-in-weight feeder at the extruder's powder inlet. The extruder had a length-to-diameter (L/D) of 25/1 and a screw diameter of 16 mm. The corotating screws of the extruder were fully modular and could hence be configured in a variety of setups. Typically, there is a conveying (i.e. transport) zone, followed by a kneading zone. And at the end, there is shaping zone. Each zone may have different screw elements.

The extruder had one kneading zone with three wide kneading disks that were positioned at a stagger angle of 30° (i.e. the angle of crest misalignment between any two directly successive kneading disks made 30° C.).

The extruder was segmented in several zones which can be heated up or cooled down individually. The powder inlet (zone 1) was not heated, while the temperature zones following the powder inlet (zones 2 and 3) were heated to a temperature of 120° C. The temperatures of zones closer to the end of the extruder (zones 4, 5 and 6) were kept (i.e. cooled) at temperatures from 55° C. to 25° C. Cooling down was beneficial to prevent that soft and sticky material is churned out (i.e. avoiding lump formation).

Water-soluble granules of good quality were obtained. The process was stable (i.e. no torque overload). Long term processibility (more than 1 hour) was achieved, indicating its potential for continuous manufacturing.

The process above was then repeated but with adding microcapsules comprising vitamin E acetate to the powderous, dry mixtures. Microcapsules were obtained from DSM® Nutritional Products (Switzerland).

Again, water-soluble granules of good quality were obtained. The process was stable (i.e. no torque overload). Long term processibility (more than 1 hour) was achieved, indicating the potential for continuous manufacturing.

1. A mixture for continuous melt granulation comprising a filler, a binder and at least one active ingredient,
wherein the mixture comprises from 5 weight-% to 15 weight-% binder, based on the total weight of the mixture, and
wherein the weight ratio between the filler and the binder is from 4:1 to 10:1, and
wherein the melting temperature of the binder is lower than the melting temperature of the filler, and
wherein the melting temperature of the filler is from 151° C. to 240° C.

2. The mixture according to claim 1, wherein the filler and the binder are non-identical polyols.

3. The mixture according to claim 1, wherein the filler and the binder are non-identical sugar alcohols.

4. The mixture according to claim 1, wherein the filler is a stereoisomer of the binder.

5. The mixture according to claim 1, wherein the filler is mannitol.

6. The mixture according to claim 1, wherein the binder is sorbitol.

7. The mixture according to claim 1 or 6, claim 1, wherein the filler comprises 2′-O-fucosyllactose.

8. The mixture according to claim 7, wherein the filler is a mixture comprising 2′-O-fucosyllactose and difucosyllactose.

9. The mixture according to claim 7, wherein said 2′-O-fucosyllactose is crystalline 2′-O-fucosyllactose.

10. The mixture according to claim 1, wherein the weight ratio between the filler and the binder is from 5:1 to 9:1, from 6:1 to 9:1, or from 7:1 to 9:1.

11. The mixture according to claim 1, wherein the mixture comprises from 0.1 weight-% to 10 weight-% of at least one water-soluble or water-dispersible vitamin, based on the total weight of the mixture, and wherein the mixture preferably comprises vitamin B1, vitamin B2, vitamin B3, vitamin B6 and/or vitamin B12.

12. The mixture according to claim 1, wherein the mixture comprises less than 10 weight-% water, preferably less than 8 weight-% water, more preferably less than 5 weight-% water and most preferably less than 3 weight-% water, based on the total weight of the mixture.

13. Granules comprising the mixture according to claim 1.

14. Granules consisting of the mixture according to claim 1.

15. The granules according to claim 13, wherein the granules have a mass median particles size D50 (volume based) from 0.5 mm to 6 mm, preferably from 1 mm to 5 mm, more preferably from 1.5 mm to 4.5 mm and most preferably from 2 mm to 4 mm, measured using dynamic image analysis.

16. The granules according to claim 13, wherein the granules are obtainable by continuous melt granulation of the mixture, preferably using a twin-screw extruder.

17. The granules according to claim 13, wherein the granules are water-soluble or water-dispersible.

18. Use of the mixture according to claim 1 for continuous melt granulation.

19. A method of manufacturing granules, comprising the step of feeding the mixture according to claim 1 into an extruder.

20. The method according to claim 19, wherein the extruder is a twin-screw extruder that has no cutting device.

21. The method according to claim 19, wherein the method does not comprise the step of cutting an extruded strand.

22. The method according to claim 19, wherein said extruder has at least one kneading zone, and wherein said at least one kneading zone has preferably at least two, more preferably at least three kneading elements that are preferably positioned at a stagger angle that is preferably from 30° to 60°.

23. The method according to claim 22, wherein the at least one kneading zone of the extruder is heated to a temperature of from 90 to 210° C., from 90 to 200° C., from 100° C. to 190° C., from 100° C. to 200° C., from 150° C. to 210° C., from 160° C. to 200° C., from 120° C. to 190° C., or from 165° C. to 190° C.

24. Granules obtained from the method according to claim 19.
